(19) United States
(12) Patent Application Publication
(10) Pub. No.: US 2024/0336558 A1
(43) Pub. Date: Oct. 10, 2024

(54) IONIZABLE LIPID BASED ON ENDOGENOUS DICARBOXYLIC ACID AS WELL AS PREPARATION METHOD AND USE THEREOF

(71) Applicant: SHANDONG UNIVERSITY, Jinan (CN)

(72) Inventors: Xinyi JIANG, Jinan (CN); Chunwei TANG, Jinan (CN); Ying LIU, Jinan (CN); Kun HAN, Jinan (CN); Kun ZHAO, Jinan (CN); Chen CHEN, Jinan (CN); Weiqiang JING, Jinan (CN); Xiaotian ZHAO, Jinan (CN); Maosen HAN, Jinan (CN)

(73) Assignee: SHANDONG UNIVERSITY, Jinan (CN)

(21) Appl. No.: 18/630,187

(22) Filed: Apr. 9, 2024

(30) Foreign Application Priority Data
Apr. 10, 2023 (CN) 2023103713921

(51) Int. Cl. C07C 233/36; A61K 9/16; A61K 31/7105; A61K 31/711; A61K 48/00

(52) U.S. Cl. CPC C07C 233/36; A61K 9/1617; A61K 31/7105; A61K 31/711; A61K 48/0033

(57) ABSTRACT

An ionizable lipid based on endogenous dicarboxylic acid as well as a preparation method and use thereof. The ionizable lipid based on endogenous dicarboxylic acid is a compound represented by Formula (I), or a stereoisomer, tautomer, pharmaceutically acceptable salt or solvate thereof;

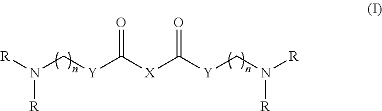

wherein, X is selected from —

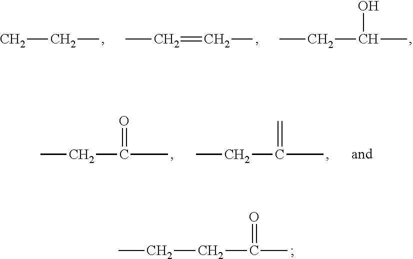

Y is selected from —O— and —NH—; R is selected from C₆-C₂₀ alkyl, C₆-C₂₀ alkyl substituted by a substituent group, C₆-C₂₀ alkenyl, C₆-C₂₀ alkenyl substituted by a substituent group, C₆-C₂₀ alkynyl, and C₆-C₂₀ alkynyl substituted by a substituent group; n is an integer of 1-8.

### TECHNICAL FIELD

The present invention belongs to the technical field of biomedicine, and relates to an ionizable lipid based on endogenous dicarboxylic acid as well as a preparation method and use thereof.

### BACKGROUND

Information disclosing the background is only intended to enhance the understanding of the overall background of the present invention, and is not necessarily considered to recognize or imply in any form that this information constitutes the existing technology that has been well-known to those skilled in the art.

Since a nucleic acid drug molecule has large molecular weight and poor own cell penetrability, it is prone to degradation by intracellular nucleases, posing significant challenges for their in-vivo applications. Therefore, development of a safe and effective nucleic acid delivery carrier to improve the ability of the nucleic acid drug to reach a target site is a key to exert the application potential of gene therapy. In the prior art, nucleic acid drugs such as nucleic acid molecule RNA are delivered to target cells through lipid nanoparticles, despite a series of ionizable lipid compounds have been reported in the prior art, but there is still a need to provide a lipid compound having efficient and stable delivery performance. Therefore, it is necessary for developing a lipid compound that is high in efficiency, small in toxicity and good in targeting.

### SUMMARY

In order to solve the defects in the prior art, the objective of the present invention is to provide an ionizable lipid based on endogenous dicarboxylic acid as well as a preparation method and use thereof. The ionizable lipid based on endogenous dicarboxylic acid provided by the present invention is used for preparing lipid nanoparticles for effective nucleic acid delivery, thereby achieving targeted gene therapy.

In order to achieve the above-mentioned objective, the technical solution of the present invention is as follows:

In one aspect, provided is an ionizable lipid based on endogenous dicarboxylic acid, wherein the ionizable lipid is a compound represented by Formula (I), or a stereoisomer, tautomer, pharmaceutically acceptable salt or solvate thereof,

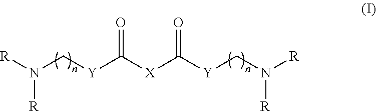

wherein, X is selected from —CH₂—CH₂—, —CH₂═CH₂—,

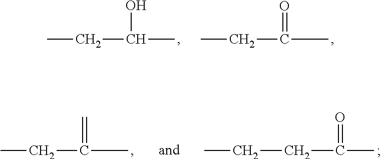

Y is selected from —O— and —NH—; R is selected from C₆-C₂₀ alkyl, C₆-C₂₀ alkyl substituted by a substituent group, C₆-C₂₀ alkenyl, C₆-C₂₀ alkenyl substituted by a substituent group, C₆-C₂₀ alkynyl, and C₆-C₂₀ alkynyl substituted by a substituent group; n is an integer of 1-8.

In another aspect, provided is a method for preparing the above-mentioned ionizable lipid based on endogenous dicarboxylic acid, comprising a step of obtaining a compound represented by Formula (I) by carrying out esterification reaction or amidation reaction on a compound represented by Formula (II) and a compound represented by Formula (III);

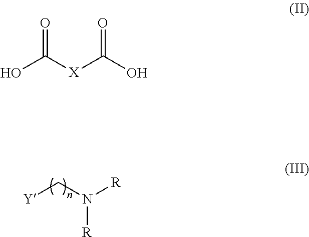

wherein, Y′ is amino or hydroxyl.

In a third aspect, provided is a composition, comprising the above-mentioned ionizable lipid based on endogenous dicarboxylic acid, a neutral lipid, a hydrophobic lipid and a polyethylene glycol (PEG)-lipid.

In a fourth aspect, provided is a use of the above-mentioned composition as a gene drug delivery carrier.

In a fifth aspect, provided is a use of the above-mentioned composition in preparing a gene drug, wherein the gene drug comprises an active ingredient which is a nucleic acid drug and a delivery carrier which is the above-mentioned composition.

The present invention has the beneficial effects:

(1) The ionizable lipid provided by the present invention based on endogenous dicarboxylic acid generated in the process of biological metabolism as a basic backbone, with an ester bond or an amido bond as linkage bonds, can be rapidly hydrolyzed in vivo by an enzyme, exhibiting excellent biocompatibility and biodegradability; this lipid compound has many hydrophobic tails so as to enhance intracellular escape and improve transfection effects; under the support of other auxiliary lipids, the ionizable lipid can effectively load nucleic acid drugs such as siRNA and mRNA to form stable and homogeneous lipid nanoparticles. Meanwhile, in terms of electric neutrality in a physiological environment, the lipid nanoparticle has better in-vivo stability, thereby reducing cytotoxicity caused by excessive positive charges; therefore, the lipid nanoparticle prepared based on the ionizable lipid compound provided by the present disclosure can be used as a safe and efficient gene delivery carrier.

(2) Relative to the traditional ionizable lipids, the ionizable lipid compound provided by the present invention is common and easily available in initial raw materials, simple in synthesis steps, mild in reaction conditions and easy in product separation, and has a good biomedicine application value.

### BRIEF DESCRIPTION OF THE DRAWINGS

The specification drawings constituting one part of the present invention are used for providing the further understanding of the present invention, the illustrative embodiments of the present invention and descriptions thereof are used for explaining the present invention, but do not constitute the improper limitation of the present invention.

FIG. 1 is a transmission electron microscopy (TEM) image of an YHS-12-1 lipid nanoparticle prepared in example 14 according to the present invention;

FIG. 2 shows positive rate results of transfection of GFP mRNA on Hep3B cells by lipid nanoparticles in different prescriptions in examples according to the present invention;

FIG. 3 shows detection results of in-vitro cytotoxicity of YHS-12-2 lipid nanoparticles in examples according to the present invention.

### DETAILED DESCRIPTION

It should be noted that the following detailed descriptions are all illustrative, and are intended to provide further description of the present invention. Unless otherwise stated, all technical and scientific terminologies used herein have the same meanings as those commonly understood by persons of ordinary skill in the art.

It is worth noting that the terminologies used here are only for describing specific embodiments, but are not intended to limit exemplary embodiments according to the present invention. As used herein, unless otherwise explicitly stated in the context, a singular form is also intended to include a plural form. In addition, it should also be understood that when the terms “comprising” and/or “including” are used in this specification, they show that there are features, steps, operations, devices, components and/or their combinations.

In view of the problems that the nucleic acid drugs such as siRNA, mRNA and ASO in the current gene therapy are sensitive to nuclease and cannot penetrate into cells, the present invention provides an ionizable lipid based on endogenous dicarboxylic acid as well as a preparation method and use thereof, in order to solve the above-mentioned technical problems.

One typical embodiment of the present invention provides an ionizable lipid based on endogenous dicarboxylic acid, which is a compound represented by Formula (I), or a stereoisomer, tautomer, pharmaceutically acceptable salt or solvate thereof.

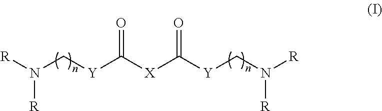

wherein, X is selected from —CH₂—CH₂—, —CH₂═CH₂—,

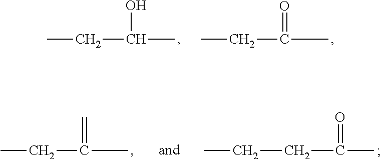

Y is selected from —O— and —NH—; R is selected from C₆-C₂₀ alkyl, C₆-C₂₀ alkyl substituted by a substituent group, C₆-C₂₀ alkenyl, C₆-C₂₀ alkenyl substituted by a substituent group, C₆-C₂₀ alkynyl, and C₆-C₂₀ alkynyl substituted by a substituent group; n is an integer of 1-8.

The substituent group is halogen or amino. The alkyl is linear alkyl or branched alkyl, preferable branched alkyl. The alkenyl is linear alkenyl or branched alkenyl, preferably branched alkenyl. The alkynyl is linear alkynyl or branched alkynyl, preferably branched alkynyl.

In some embodiments, X is selected from —CH₂—CH₂—, —CH═CH—, and

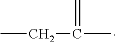

In some embodiments, Y is —NH—.

In some embodiments, R is C₆-C₂₀ alkyl.

In some embodiments, n is an integer of 1-3.

Specifically, compounds are as follows:

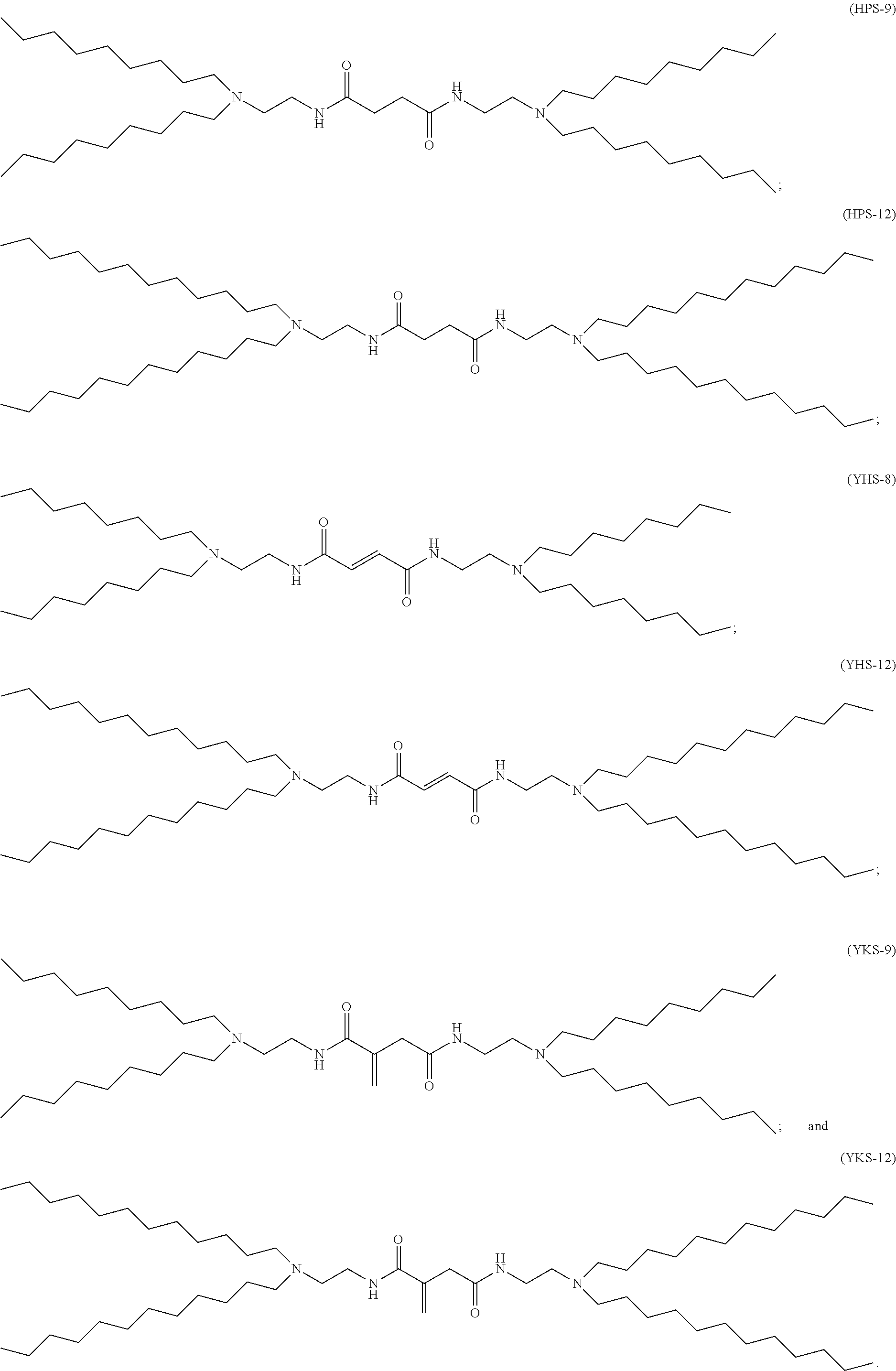

Another embodiment of the present invention provides a method for preparing the above-mentioned ionizable lipid based on endogenous dicarboxylic acid, comprising a step of obtaining a compound represented by Formula (I) by carrying out esterification reaction or amidation reaction on a compound represented by Formula (II) and a compound represented by Formula (III);

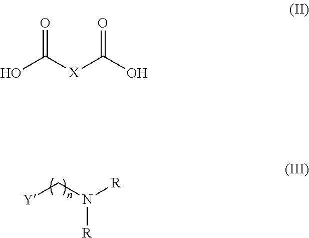

wherein, Y′ is amino or hydroxyl.

In some embodiments, the esterification reaction is carried out in the presence of 1-(3-dimethylaminopropyl)-3-ethylcarbodiimide hydrochloride and 1-hydroxybenzotriazole.

In some embodiments, the amidation reaction is carried out in ice bath.

In some embodiments, the compound represented by Formula (II) comprises succinic acid, fumaric acid, itaconic acid, α-ketoglutaric acid, oxaloacetic acid, and L-malic acid.

The third embodiment of the present invention provides a composition, comprising the above-mentioned ionizable lipid based on endogenous dicarboxylic acid, a neutral lipid, a hydrophobic lipid and a PEG-lipid.

In some embodiments, the composition constitutes lipid nanoparticles. Specifically, the lipid nanoparticles are positively charged at pH 4.0, and uncharged or negatively charged at pH 7.0. The method for preparing the lipid nanoparticles comprises a lipid extrusion method, a film hydration method, a nano precipitation method, a microfluidic and impact jet flow mixing method and the like, preferably the nano precipitation method.

In one or more embodiments, the particle size of the lipid nanoparticle is 1-1000 nm, preferably 100-200 nm.

In some embodiments, a molar ratio of the ionizable lipid based on endogenous dicarboxylic acid to the neutral lipid to the hydrophobic lipid to the PEG-lipid is (2-60):(5-55):(10-50):(0.2-25); preferably is (15-45):(10-40):(20-35):(0.5-5).

In some embodiments, the neutral lipid comprises distearoyl phosphatidylcholine (DSPC), dipalmitoyl phosphatidylcholine (DPPC), dipalmitoyl phosphatidylglycerol (DPPG), dioleoyl phosphatidylglycerol (DOPG), dioleoyl phosphatidylcholine (DOPC), dioleoyl phosphatidylethanolamine (DOPE), phosphatidylcholine (POPC), palmitoyl oleyl phosphatidylethanolamine (POPE), dilauroyl phosphatidylcholine (DLPC), diethyl pyrocarbonate (DEPC), dimyristoyl phosphatidylcholine (DMPC), egg lecithin (EPC), hydrogenated soy phosphatidylcholine (HSPC), and sphingomyelin (SM). Preferably, the neutral lipid is DOPE.

In some embodiments, the hydrophobic lipid is sterol. The sterol is cholesterol.

In some embodiments, the PEG-lipid comprises distearoyl phosphoethanolamine-PEG (DSPE-PEG), dipalmitoyl phosphoethanolamine-PEG (DPPE-PEG), dimyristoyl glycerol-PEG (DMG-PEG) or dimethyl acrylate-PEG (DMA-PEG). Preferably, the PEG-lipid is DMG-PEG.

In some embodiments, the composition comprises a buffer reagent. The buffer reagent is preferably an acidic buffer reagent. The buffer reagent comprises citric acid, sodium citrate, disodium hydrogen phosphate, sodium dihydrogen phosphate, acetic acid, sodium acetate, trihydroxymethylaminomethane-hydrochloric acid, potassium dihydrogen phosphate-sodium hydroxide, glycine-hydrochloric acid, boric acid-borax, phthalic acid-hydrochloric acid, potassium hydrogen phthalate, and sodium dihydrogen phosphate-citric acid. The buffer reagent is preferably citric acid. Specifically, when the lipid nanoparticles are formed, the volume fraction of the buffer reagent is 40-90% v/v, preferably 75% v/v.

In some embodiments, the composition comprises an organic reagent. The organic reagent is selected from one or more of methanol, ethanol, isopropanol, benzene, toluene, xylene, pentane, hexane, octane, cyclohexane, cyclohexanone, toluene cyclohexanone, chlorobenzene, dichlorobenzene, ether, dichloromethane, epoxypropane, acetone, methyl butanone, methyl isobutanone, acetonitrile, pyridine, phenol, styrene, perchloroethylene, trichloroethylene, ethylene glycol ether, and triethanolamine, preferably ethanol.

The fourth embodiment of the present invention provides a use of the above-mentioned composition as a gene drug delivery carrier.

The fifth embodiment of the present invention provides a use of the above-mentioned composition in preparing a gene drug, wherein the gene drug comprises an active ingredient which is a nucleic acid drug and a delivery carrier which is the above-mentioned composition.

In some embodiments, the nucleic acid drug is siRNA, mRNA, tRNA, rRNA, cDNA, ASO, plasmid DNA, microRNA, long non-coding RNA, and the like, preferably mRNA. The mRNA comprises straight-stranded mRNA and circular mRNA.

In some embodiments, a mass ratio of the active ingredient to the delivery carrier is 1:8-12.

In order to make those skilled in the art more clearly know the technical solution of the present invention, the technical solution of the present invention will be described in detail in conjunction with specific embodiments below.

### Example 1 Synthesis of 1,1-dimethylethyl-N-[2-(dinonylamino)ethyl]carbamate

N-tert-butoxycarbonyl-1,2-ethylenediamine (1.76 g, 10 mmol), 1-bromononane (5.00 g, 22 mmol), potassium carbonate (3.03 g, 20 mmol) and anhydrous acetonitrile (30 ml) were added into a 250 mL round-bottom flask equipped with magnetons. A condensing pipe was installed on the reaction bottle. The above-mentioned materials were subjected to heating reflux for 72 h at 80° C. The reaction solution was subjected to suction filtration, a solid was discarded, and a solvent was removed by vacuum rotary evaporation. Subsequently, the obtained product was separated and purified via a thin layer chromatography column (a volume ratio of an eluent to methanol to dichroomethane=1:20) to give an intermediate product 1,1-dimethylethyl-N-[2-(dinonylamino)ethyl] carbamate, with a yield of 83%. The specific reaction equation was as follows:

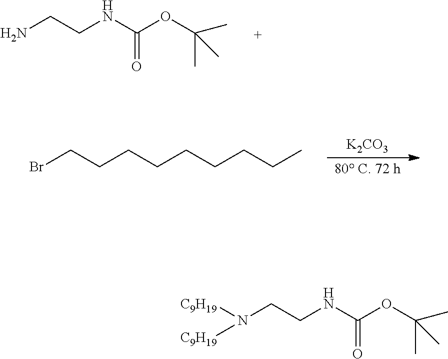

### Example 2 Synthesis of N¹,N¹-dinonyl-1,2-ethylenediamine

1,1-dimethylethyl-N-[2-(dinonylamino)ethyl] carbamate (4.00 g, 9.7 mmol) was added into a 100 mL round-bottom flask equipped with magnetons and dissolved with 1,4-dioxane (25 mL) followed by a 1,4-dioxane hydrochloride solution (25 mL), and then the above-mentioned materials were stirred for 8 h at room temperature. The reaction solution was subjected to vacuum rotary evaporation, an appropriate amount of saturated sodium bicarbonate solution was added, the mixed solution was extracted with dichloromethane (3×30 mL), an organic phase was dried with anhydrous sodium sulfate and filtrated, and a solvent was removed by using a rotary evaporator, so as to give a crude product of N¹,N¹-dinonyl-1,2-ethylenediamine. The crude product participated in a next-step reaction without purification. The specific reaction equation was as follows:

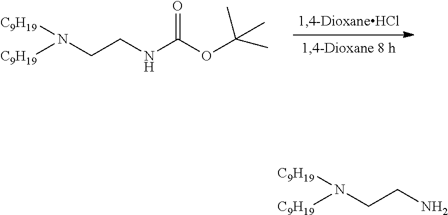

### Example 3 Synthesis of (2-(didodecylamino) ethyl) tert-butyl carbamate

N-tert-butoxycarbonyl-1,2-ethylenediamine (1.8 g, 11 mmol), 1-bromododecane (6.0 g, 24 mmol), potassium carbonate (3.04 g, 22 mmol) and anhydrous acetonitrile (40 ml) were added into a 250 mL round-bottom flask equipped with magnetons. A condensing pipe was installed on the reaction bottle. The above-mentioned materials were subjected to heating reflux for 72 h at 80° C. The reaction solution was subjected to suction filtration, a solid was discarded, and a solvent was removed by vacuum rotary evaporation. The obtained product was separated and purified via a thin layer chromatography column (a volume ratio of an eluent to methanol to dichroomethane=1:20), so as to give an intermediate product (2-(didodecylamino) ethyl) tert-butyl carbamate, with a yield of 78%. The specific reaction equation was as follows:

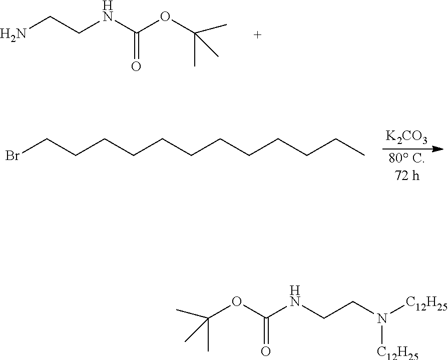

### Example 4 Synthesis of N¹,N¹-dodecyl ethane-1,2-diamine

(2-(didodecylamino)ethyl) tert-Butyl carbamate (4.0 g, 8 mmol) was added into a 100 mg round-bottom flask equipped with magnetons and dissolved with 1,4-dioxane (20 mL) followed by adding a 1,4-dioxane hydrochloride solution (20 mL). The above-mentioned materials were stirred for 6 h at room temperature. The reaction solution was subjected to vacuum rotary evaporation, an appropriate amount of saturated sodium bicarbonate solution was added, the above-mentioned mixed solution was extracted with dichloromethane (3×30 mL), an organic phase was dried with anhydrous sodium sulfate and filtrated, and a solvent was removed by using a rotary evaporator to give a crude product of N¹,N¹-didodecyl ethane-1,2-diamine. The crude product participated in a next-step reaction without purification. The specific reaction equation was as follows:

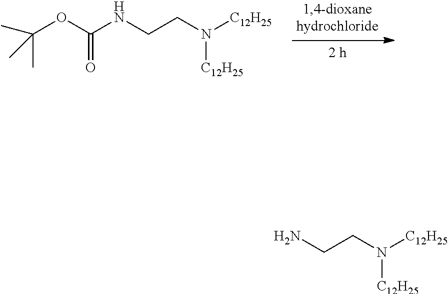

### Example 5 Synthesis of 1,1-dimethylethyl-N-[2-(dioctylamino)ethyl] carbamate

N-tert-butoxycarbonyl-1,2-ethylenediamine (1.92 g, 12 mmol), 1-bromooctane (5.00 g, 26 mmol), potassium carbonate (3.32 g, 24 mmol) and anhydrous acetonitrile (30 ml) were added into a 250 mL round-bottom flask equipped with magnetons. A condensing pipe was installed on the reaction bottle. The above-mentioned materials were subjected to heating reflux for 72 h at 80° C. The reaction solution was subjected to suction filtration, a solid was discarded, and a solvent was removed by vacuum rotary evaporation. The obtained product was separated and purified via a thin layer chromatography column (a volume ratio of an eluent to methanol to dichroomethane=1:20), so as to give an intermediate product 1,1-dimethylethyl-N-[2-(dioctylamino)ethyl] carbamate, with a yield of 79%. Specific reaction equation was as follows:

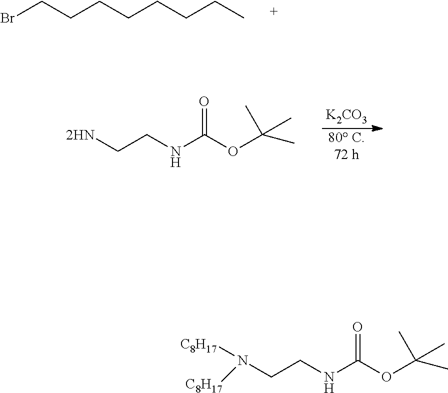

### Example 6 Synthesis of N¹,N¹-dioctyl-1,2-ethylenediamine

1,1-dimethylethyl-N-[2-(dioctylamino)ethyl] carbamate (2.00 g, 5.2 mmol) was added into a 100 mL round-bottom flask equipped with magnetons and dissolved with 1,4-dioxane (13 mL) followed by adding a 1,4-dioxane hydrochloride solution (13 mL). The above-mentioned materials were stirred for 6 h at room temperature. The reaction solution was subjected to vacuum rotary evaporation. An appropriate amount of saturated sodium bicarbonate solution was added. The mixed solution was extracted with dichloromethane (3×30 mL), an organic phase was dried with anhydrous sodium sulfate and filtrated, and a solvent was removed by using a rotary evaporator, so as to give a crude product of N¹,N¹-dioctyl-1,2-ethylenediamine. The crude product participated in a next-step reaction without purification. The specific reaction equation was as follows:

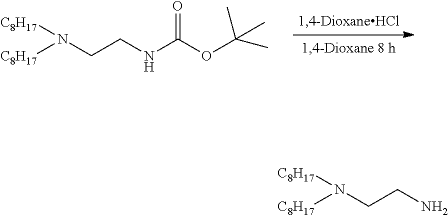

### Example 7 Synthesis of Itaconyl Chloride

Itaconyl chloride (1.00 g, 7.7 mmol) and tripe amounts of oxalyl chloride (23.1 mmol, 1.97 ml) were added into a 100 mL round-bottom flask equipped with magnetons and dissolved with dichloromethane (25 mL) followed by adding 3-5 drops of dimethyl formamide (DMF). The above-mentioned materials were stirred for 6 h at room temperature. Note: an exhaust gas treatment device must be connected, and backsuction is prevented. The reaction solution was subjected to vacuum rotary evaporation to give a crude product of itaconyl chloride. The crude product participated in a next-step reaction without purification. The specific reaction equation was as follows:

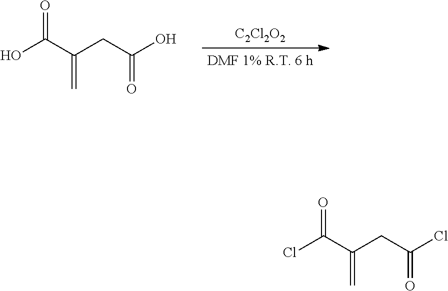

### Example 8 Synthesis of N¹,N⁴-bis(2-(dinonylamino)ethyl)-2-methylene succinamide

The prepared N¹,N¹-dinonyl-1,2-ethylenediamine (1.0 g, 3.20 mmol) was added into a 100 mL round-bottom flask equipped with magnetons and dissolved with anhydrous dichloromethane (10 mL); the prepared itaconyl chloride (0.24 g, 1.45 mmol) was taken and dissolved with anhydrous dichloromethane, and then the dissolved itaconyl chloride was added into a constant-pressure dropping funnel and slowly dropwise added into a solution of N¹,N¹-dinonyl-1,2-ethylenediamine in dichloromethane in ice bath, wherein the drip rate was controlled until dropwise addition was completed within 1 h, followed by continuously stirring for 24 h. The reaction solution was directly subjected to vacuum drying without posttreatment. The residual mixture was separated by silica gel column chromatography (a volume ratio of eluent to methanol to dichloromethane=1:10) to give 76 mg of target product as a yellow oily liquid, with a yield of 7.31%. ¹H NMR (400 MHz, Chloroform-d) δ 6.33 (d, J=2.5 Hz, 1H), 5.60 (d, J=2.2 Hz, 1H), 3.65 (t, J=6.8 Hz, 4H), 3.29 (s, 2H), 2.63 (t, J=6.8 Hz, 3H), 2.45-2.37 (m, 8H), 1.25 (s, 56H), 0.88 (t, J=6.7 Hz, 12H). The specific reaction equation was as follows:

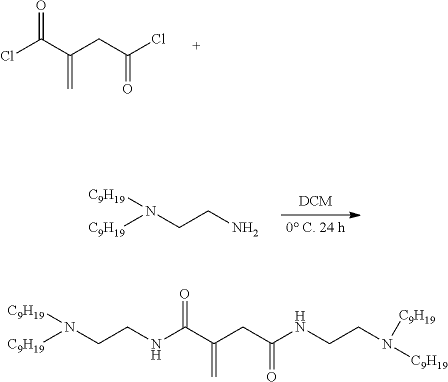

### Example 9 Synthesis of N¹,N⁴-bis(2-(didodecylamino)ethyl)-2-methylene succinamide

The prepared N¹,N¹-didodecylethane-1,2-ethylenediamine (1.0 g, 2.52 mmol) was added into a 100 mL round-bottom flask equipped with magnetons and dissolved with anhydrous dichloromethane (10 mL); the prepared itaconyl chloride (0.19 g, 1.15 mmol) was dissolved with anhydrous dichloromethane, and then the dissolved itaconyl chloride was added into a constant-pressure dropping funnel and slowly dropwise added into a solution of N¹,N¹-didodecylethane-1,2-ethylenediamine in ice bath, wherein a drip rate was controlled until dropwise addition was completed within 1 h, followed by continuously stirring for 24 h. The reaction solution was directly subjected to vacuum drying without posttreatment. The residual mixture was separated by silica gel column chromatography (a volume ratio of eluent to methanol to dichloromethane=1:10) to give 84 mg of target product as a yellow oily liquid, with a yield of 10%. ¹H NMR (400 MHz, Chloroform-d) δ 6.25 (d, J=2.5 Hz, 1H), 5.52 (d, J=2.2 Hz, 1H), 3.58 (t, J=6.8 Hz, 4H), 3.23-3.21 (m, 2H), 2.56 (t, J=6.8 Hz, 3H), 2.40-2.27 (m, 8H), 1.18 (s, 80H), 0.82 (d, J=6.6 Hz, 12H). The specific reaction equation was as follows:

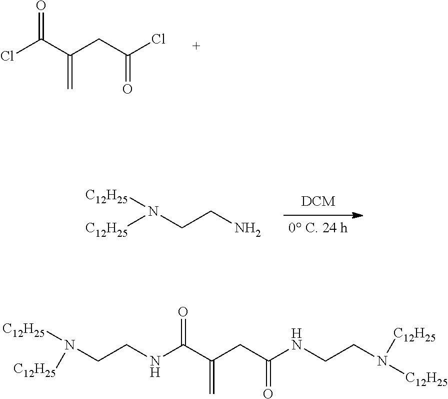

### Example 10 Synthesis of N¹,N⁴-bis(2-(dinonylamino)ethyl) succinamide

The prepared N¹,N¹-dinonyl-1,2-ethylenediamine (1.0 g, 3.20 mmol) was added into a 100 mL round-bottom flask equipped with magnetons and dissolved with anhydrous dichloromethane (10 mL); succinyl chloride (0.22 g, 1.45 mmol) was taken and dissolved with anhydrous dichloromethane, and then the dissolved succinyl chloride was added into a constant-pressure dropping funnel and slowly dropwise added into a solution of N¹,N¹-dinonyl-1,2-ethylenediamine in dichloromethane in ice bath, wherein a drip rate was controlled until dropwise addition was completed within 1 h, followed by continuously stirring for 24 h. The reaction solution was directly subjected to vacuum drying without posttreatment. The residual mixture was separated by silica gel column chromatography (a volume ratio of eluent to methanol to dichloromethane=1:10) to give 53 mg of target product as a yellow oily liquid, with a yield of 5.13%. ¹H NMR (600 MHz, Chloroform-d) δ 3.51 (t, J=6.8 Hz, 4H), 2.53 (dd, J=14.5, 7.6 Hz, 8H), 2.33 (t, J=7.4 Hz, 8H), 1.30-1.18 (m, 56H), 0.82 (d, J=6.8 Hz, 12H). The specific reaction equation was as follows:

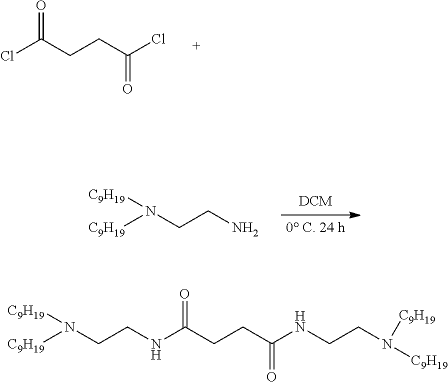

### Example 11 Synthesis of N¹,N⁴-bis(2-(didodecylamine)ethyl) succinamide

The prepared N¹,N¹-didodecylethane-1,2-ethylenediamine (1.0 g, 2.52 mmol) was added into a 100 mL round-bottom flask equipped with magnetons and dissolved with anhydrous dichloromethane (10 mL); succinyl chloride (0.18 g, 1.15 mmol) was taken and dissolved with anhydrous dichloromethane, and then the dissolved succinyl chloride was added into a constant-pressure dropping funnel and slowly dropwise added into a solution of N1,N1-didodecylethane-1,2-ethylenediamine in dichloromethane in ice bath, wherein a drip rate was controlled until dropwise addition was completed within 1 h, followed by continuously stirring for 24 h. The reaction solution was directly subjected to vacuum drying without posttreatment. The residual mixture was separated by silica gel column chromatography (a volume ratio of eluent to methanol to dichloromethane=1:10) to give 64 mg of target product as a yellow oily liquid, with a yield of 6.4%. ¹H NMR (400 MHz, Chloroform-d) δ 3.51 (t, J=6.8 Hz, 4H), 2.61-2.49 (m, 8H), 2.37-2.30 (m, 7H), 1.19 (s, 80H), 0.81 (t, J=6.7 Hz, 12H). The specific reaction equation was as follows:

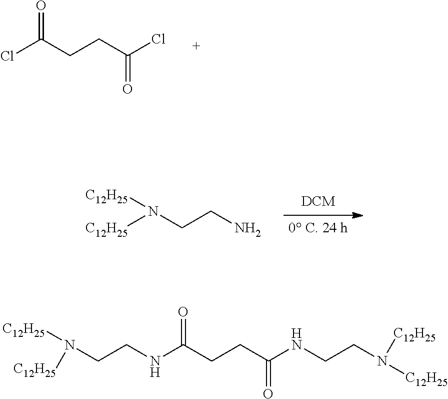

### Example 12 Synthesis of N¹,N⁴-bis(2-(dioctylamino)ethyl) fumaramide

The prepared N¹,N¹-dioctyl-1,2-ethylenediamine (0.5 g, 1.76 mmol) was added into a 100 mL round-bottom flask equipped with magnetons and dissolved with anhydrous dichloromethane (10 mL); fumaryl chloride (0.12 g, 0.84 mmol) was taken and dissolved with anhydrous dichloromethane, and then the dissolved fumaryl chloride was added into a constant-pressure dropping funnel and slowly dropwise added into a solution of N¹,N¹-dioctyl-1,2-ethylenediamine in dichloromethane in ice bath, wherein a drip rate was controlled until dropwise addition was completed within 1 h, followed by continuously stirring for 24 h. The reaction solution was directly subjected to vacuum drying without posttreatment. The residual mixture was separated by silica gel column chromatography (a volume ratio of eluent to methanol to dichloromethane=1:10) to give 30 mg of target product as a white solid, with a yield of 5.45%. ¹H NMR (400 MHz, Chloroform-d) δ 6.89 (s, 2H), 6.82 (t, J=5.1 Hz, 2H), 3.31 (q, J=5.8 Hz, 4H), 2.51 (t, J=6.1 Hz, 4H), 2.39-2.23 (m, 8H), 1.37-1.13 (m, 48H), 0.81 (t, J=6.7 Hz, 12H). The specific reaction equation was as follows:

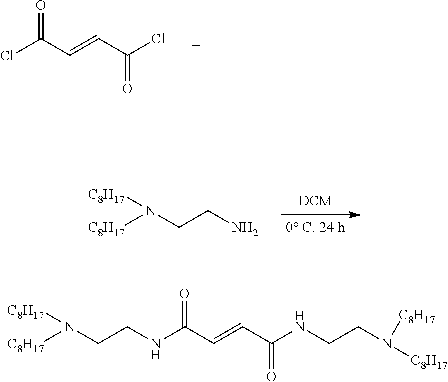

### Example 13 Synthesis of N¹,N⁴-bis(2-(didodecylamino)ethyl) fumaramide

The prepared N¹,N¹-didodecylethane-1,2-ethylenediamine (0.6 g, 1.51 mmol) was added into a 100 mL round-bottom flask equipped with magnetons and dissolved with anhydrous dichloromethane (10 mL); fumaryl chloride (0.11 g, 0.72 mmol) was dissolved with anhydrous dichloromethane, and then the dissolved fumaryl chloride was added into a constant-pressure dropping funnel and slowly dropwise added into a solution of N¹,N¹-didodecylethane-1,2-ethylenediamine in dichloromethane in ice bath, wherein a drip rate was controlled until dropwise addition was completed within 1 h, followed by continuously stirring for 24 h. The reaction solution was directly subjected to vacuum drying without posttreatment. The residual mixture was separated by silica gel column chromatography (a volume ratio of eluent to methanol to dichloromethane=1:10) to give 155 mg of target product as a white solid, with a yield of 24.6%. ¹H NMR (600 MHz, Chloroform-d) δ 6.83 (s, 2H), 6.65 (s, 2H), 3.32 (q, J=5.8 Hz, 4H), 2.51 (t, J=6.0 Hz, 4H), 2.35 (t, J=7.6 Hz, 8H), 1.37-1.15 (m, 80H), 0.81 (t, J=7.0 Hz, 12H). The specific reaction equation was as follows:

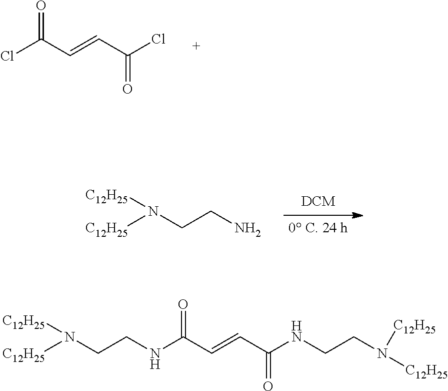

### Example 14 Preparation and Characterization of Ionizable Lipid Nanoparticles

The lipid YHS-12 prepared in example 13 was selected as a representative compound to prepare lipid nanoparticles. First, the ionizable lipid (YHS-12) for preparing lipid nanoparticles, DSPC, cholesterol and DMG-PEG were respectively set to 4 levels, as shown in Table 1. Subsequently, 16 nanoparticle prescriptions were optimized and designed by using orthogonal design software. Molar ratios and mass ratios of different components in the 16 prescriptions are shown in Table 2. YHS-12, DOPE, cholesterol and DMG-PEG were respectively dissolved into anhydrous ethanol to prepare 10 mg/ml stock solutions. After the above-mentioned materials were prepared, four raw material stock solutions were mixed according to the mass ratios in Table 2, and completely sucked into an insulin syringe for later use. In addition, mRNA (purchased from carrier's EGFP IVT mRNA, ml Ψ modification, a mass ratio of mRNA to YHS-12 was 1:10) was dissolved into a citric acid buffer that has a pH of 4.0 and a concentration of 50 mM and whose volume was as three times as that of an organic phase mixed solution. Similarly, the mixed solution was completely sucked into an insulin syringe for later use. After that, the insulin syringes with an organic phase mixed solution and a water phase buffer were respectively placed in the same centrifuge tube and all the solution was rapidly released. The centrifuge tube was slightly and evenly shaken to prepare the lipid nanoparticles. Finally, the product underwent dialysis at 4° C. overnight in PBS buffer. In the 16 prescriptions, the lipid nanoparticles were prepared according to the above-mentioned method, which were named YHS-12-1, YHS-12-2, YHS-12-3, YHS-12-4, YHS-12-5, YHS-12-6, YHS-12-7, YHS-12-8, YHS-12-9, YHS-12-10, YHS-12-11, YHS-12-12, YHS-12-13, YHS-12-14, YHS-12-15, and YHS-12-16.

TABLE 1

| YHS-12 | Cholesterol | DOPE | DMG-PEG |
|---|---|---|---|
| 15 | 20 | 10 | 0.5 |
| 25 | 25 | 20 | 1 |
| 35 | 30 | 30 | 2.5 |
| 45 | 35 | 40 | 5 |

TABLE 2

| | Molar ratio | | | | Mass ratio | | | |
|---|---|---|---|---|---|---|---|---|
| Number | YHS-12 | Cholesterol | DOPE | DMG-PEG | YHS-12 | Cholesterol | DOPE | DMG-PEG |
| YHS-12-1 | 15 | 20 | 10 | 0.5 | 44.4% | 26.2% | 4.2% | 25.2% |
| YHS-12-2 | 15 | 25 | 20 | 1 | 32.6% | 24.1% | 6.2% | 37.1% |
| YHS-12-3 | 15 | 30 | 30 | 2.5 | 24.6% | 21.8% | 11.8% | 41.9% |
| YHS-12-4 | 15 | 35 | 40 | 5 | 19.0% | 19.6% | 18.2% | 43.2% |
| YHS-12-5 | 25 | 20 | 10 | 0.5 | 57.1% | 20.2% | 3.3% | 19.4% |
| YHS-12-6 | 25 | 25 | 20 | 1 | 44.7% | 19.8% | 5.1% | 30.4% |
| YHS-12-7 | 25 | 30 | 30 | 2.5 | 35.2% | 18.7% | 10.1% | 36.0% |
| YHS-12-8 | 25 | 35 | 40 | 5 | 28.1% | 17.4% | 16.2% | 38.3% |
| YHS-12-9 | 35 | 20 | 10 | 0.5 | 65.0% | 16.5% | 2.7% | 15.8% |
| YHS-12-10 | 35 | 25 | 20 | 1 | 53.1% | 16.8% | 4.4% | 25.8% |
| YHS-12-11 | 35 | 30 | 30 | 2.5 | 43.2% | 16.4% | 8.9% | 31.5% |
| YHS-12-12 | 35 | 35 | 40 | 5 | 35.4% | 15.7% | 14.5% | 34.4% |
| YHS-12-13 | 45 | 20 | 10 | 0.5 | 70.5% | 13.9% | 2.3% | 13.3% |
| YHS-12-14 | 45 | 25 | 20 | 1 | 59.2% | 14.6% | 3.8% | 22.4% |
| YHS-12-15 | 45 | 30 | 30 | 2.5 | 49.4% | 14.6% | 7.9% | 28.1% |
| YHS-12-16 | 45 | 35 | 40 | 5 | 41.3% | 14.2% | 13.2% | 31.3% |

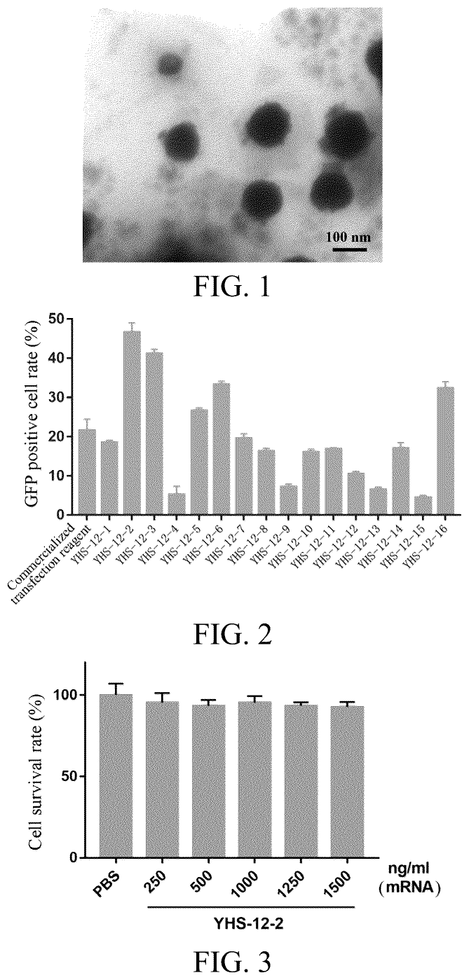

The nano sizes and polydispersity indexes PDI of lipid nanoparticles in different prescriptions were detected by using Malverm Zetasizer Nano ZS and utilizing dynamic light scattering in a 900 backscatter detection mode. The entrapment rate of the lipid nanoparticles was determined by using Quant-iT RiboGreen RNA Assay Kit. The results are shown in Table 3, indicating that the particle sizes of the 16 lipid nanoparticles are homogeneous (PDI<0.3), which are between 100 nm and 200 nm, and the lipid nanoparticles in different prescriptions all have relatively high entrapment rates on mRNA. Furthermore, the morphology of YHS-12-1 as a representative lipid nanoparticle was investigated through TEM. The results show that the lipid nanoparticle is quasi spherical, as shown in FIG. 1.

TABLE 3

| | | | Entrapment |
|---|---|---|---|
| Number | Particle size (nm) | PDI | efficiency |
| YHS-12-1 | 112.3 ± 0.35 | 0.204 ± 0.012 | 85% |
| YHS-12-2 | 151.4 ± 0.54 | 0.119 ± 0.006 | 92% |
| YHS-12-3 | 185.6 ± 1.32 | 0.132 ± 0.024 | 91% |
| YHS-12-4 | 158.4 ± 0.67 | 0.236 ± 0.018 | 79% |
| YHS-12-5 | 129.5 ± 0.89 | 0.143 ± 0.028 | 84% |
| YHS-12-6 | 117.1 ± 1.23 | 0.127 ± 0.007 | 90% |
| YHS-12-7 | 145.2 ± 0.38 | 0.146 ± 0.042 | 75% |
| YHS-12-8 | 109.2 ± 0.71 | 0.101 ± 0.015 | 83% |
| YHS-12-9 | 166.4 ± 1.52 | 0.168 ± 0.022 | 86% |
| YHS-12-10 | 125.6 ± 0.98 | 0.252 ± 0.004 | 85% |
| YHS-12-11 | 147.9 ± 0.69 | 0.162 ± 0.009 | 90% |
| YHS-12-12 | 172.3 ± 1.82 | 0.178 ± 0.014 | 88% |
| YHS-12-13 | 132.4 ± 0.18 | 0.137 ± 0.013 | 82% |
| YHS-12-14 | 101.6 ± 0.22 | 0.213 ± 0.003 | 76% |
| YHS-12-15 | 129.9 ± 2.43 | 0.133 ± 0.026 | 82% |
| YHS-12-16 | 130.6 ± 0.37 | 0.153 ± 0.015 | 86% |

### Example 15 mRNA In-Vitro Transfection Experiment of YHS-12 Lipid Nanoparticles

In this experiment, GFP-mRNA was used as model mRNA. The mRNA transfection efficiencies of 16 prescription preparations were detected in Hep3B cells, and specific operation method was as follows: the Hep3B cells in a logarithmic phase were inoculated onto a 6-well plate and cultured overnight in a cell incubator, subsequently 16 lipid nanoparticles (a final mRNA amount was 2 μg) were respectively incubated with the Hep3B cells, and a proportion of GFP positive cells was detected by flow cytometry after culture for 24 h. A commercialized gene transfection reagent, i.e., LipoSmart mRNA Transfection Reagent, was used as positive control. It can be seen from results in FIG. 2 that the lipid nanoparticles prepared by the YHS-12 compound of the present disclosure can transfect mRNA expressing GFP to the Hep3B cells, especially, the transfection efficiencies of YSH-12-2, YSH-12-3, YSH-12-5, YSH-12-6 and YSH-12-16 can be up to or more than that of the commercialized gene transfection reagent, i.e., LipoSmart mRNA Transfection Reagent, wherein the transfection efficiency of YHS-12-2 was optimal.

### Example 16 In-Vitro Cytotoxicity Analysis on Lipid Nanoparticles

To detect the cytotoxicity of the lipid nanoparticles, Hep3B cells in a logarithmic growth phase were inoculated on a 96-well plate and cultured overnight in a cell incubator, subsequently YSH-12-2 respectively having different mRNA concentrations (250, 500, 1000, 1250, and 1500 ng/ml) was incubated with the Hep3B cells, and then cell activity was detected by CCK-8 kit. The results show that under all the concentrations, the activity of Hep3B cells does not significantly change (FIG. 3), which indicates that the YHS-12-2 lipid nanoparticles provided by the present invention has lower cytotoxicity, and has excellent biocompatibility.

The above-mentioned descriptions are only preferred embodiments of the present invention, but are not intended to limit the present invention. For those skilled in the art, various changes and variations can be made to the present invention. Any modifications, equivalent replacements, improvements and the like made within the spirit and principle of the present invention all should be included within the scope of protection of the present invention.

1-8. (canceled)

9. An ionizable lipid based on endogenous dicarboxylic acid, the ionizable lipid being a compound represented by Formula (I), or a stereoisomer, tautomer, pharmaceutically acceptable salt or solvate thereof; 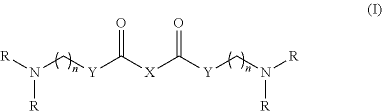
wherein, X is —CH₂═CH₂—; Y is —NH—; R is C₆-C₂₀ alkyl; n is an integer of 1-8.

10. The ionizable lipid based on endogenous dicarboxylic acid according to claim 9, comprising the following compounds:

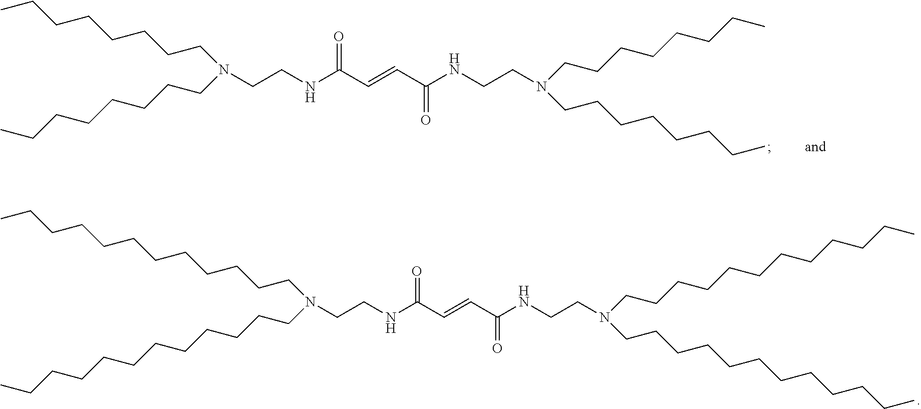

11. A method for preparing the ionizable lipid based on endogenous dicarboxylic acid according to claim 9, comprising a step of obtaining a compound represented by Formula (I) by carrying out esterification reaction or amidation reaction on a compound represented by Formula (II) and a compound represented by Formula (III); 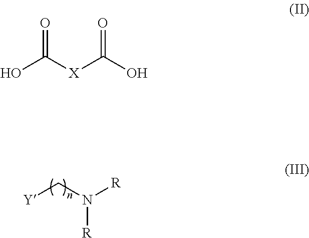
wherein, Y′ is amino, and X is consistent with the X group in claim 9.

12. A composition, comprising the ionizable lipid based on endogenous dicarboxylic acid according to claim 9, a neutral lipid, a hydrophobic lipid, and a polyethylene glycol (PEG)-lipid.

13. The composition according to claim 12, wherein the composition constitutes lipid nanoparticles.

14. The composition according to claim 13, wherein a molar ratio of the ionizable lipid based on endogenous dicarboxylic acid to the neutral lipid to the hydrophobic lipid to the PEG-lipid is (2-60):(5-55):(10-50):(0.2-25).

15. A gene drug comprising an active ingredient which is a nucleic acid drug, and a delivery carrier which is the composition according to claim 12.

16. The gene drug according to claim 15, wherein the nucleic acid drug is siRNA, mRNA, tRNA, rRNA, cDNA, ASO, plasmid DNA, microRNA, or long non-coding RNA.
